(19) United States
(12) Patent Application Publication
(10) Pub. No.: US 2008/0167599 A1
(43) Pub. Date: Jul. 10, 2008

(54) Active applicator

(75) Inventors: Thomas Ward Osborn, Cincinnati, OH (US); Diana Lynne Gann, Lebanon, OH (US); Devin William Baldridge, Cincinnati, OH (US); Margaret Henderson Hasse, Wyoming, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Comapny

(21) Appl. No.: 11/651,746

(22) Filed: Jan. 10, 2007

(51) Int. Cl. A61M 37/00

(52) U.S. Cl. 604/14

(57) ABSTRACT

An applicator for inserting an object into a cavity. The applicator can have a hollow insertion member. The applicator can further have an indicator member circumscribing or partially circumscribing the periphery of the insertion member. The indicator member can have a first position and a second position.

### FIELD OF THE INVENTION

The present invention relates to an applicator for inserting an object into a cavity.

### BACKGROUND OF THE INVENTION

There is a need for people to be able precisely place objects into cavities, such as body cavities, in situations in which the outer boundaries of the cavity may not be observable by the person inserting the object, the outer boundary of the cavity is deformable, or the amount of pressure exerted on the boundaries of the cavity and/or portions of any structure within the cavity is critical. In these situations a person can use her sense of touch to determine if the object is properly placed. Alternatively, there are a variety of applicators that can be used by people to help them properly place objects in cavities.

Tampon applicators are commonly used for inserting a tampon into a vaginal cavity to manage vaginal discharge. Similar applicators are also used for inserting pessaries, contraceptive devices, medicinal delivery devices, and the like into the vaginal cavity of the human body. Other cavities of the body into which objects are inserted include the rectum, ear canals, nasal passageways, surgical openings, and wounds.

In a simple embodiment, an applicator comprises a pair of coaxial hollow cylinders or tubes. The larger outer tube serves as an insertion tube that includes the object to be inserted and the smaller inner tube, often referred to as the plunger, is used to eject the object from the outer tube.

For many objects inserted into human body cavities, the object needs to be precisely located if the object is to perform as intended. In the art of tampons, many investigators recognize that “push” type tampon applicators have many deficiencies. Many investigators recognize that a tampon must be precisely placed to perform optimally. For example, conventional “push” type tampon applicators can place a tampon too high in the vaginal cavity above the major path of fluid flow resulting in menstrual fluid bypassing the tampon without being absorbed. Alternatively, tampon applicators can place a tampon too low in the vaginal cavity, which can result in discomfort to the wearer or the tampon being accidentally discharged from the vagina. For devices such as pessaries, the pessary needs to be properly placed to produce satisfactory results. To overcome these potential problems, some applicators have visual cues on the insertion tube and/or plunger to aid the user in properly placing the object.

For objects inserted into human body cavities, the person inserting the object may not be able to conveniently view the cavity, or boundaries thereof, into which the object is being inserted or may not want to view the cavity into which the device is being inserted. Users of tampons often have difficulty in clearly observing their crotch region and may be unable to observe cues on the tampon applicator designed to alert the user that the applicator is properly inserted.

The problem remains with providing an applicator for inserting devices into a cavity that provides a tactile signal that the applicator is properly placed. The problem also remains with providing an applicator for inserting an object into a cavity that is comfortable to use.

### SUMMARY OF THE INVENTION

An applicator for inserting an object into a cavity is disclosed. The applicator can comprise a hollow insertion member. The insertion member has an insertion end and an opposing gripper end, a length, and a periphery. The length of the insertion member is defined by the distance between the insertion end and the gripper end. The periphery about the insertion member is measured generally orthogonal to the length of the insertion member. The applicator can further comprise an indicator member circumscribing or partially circumscribing the periphery of the insertion member. The indicator member can comprise a hollow open ended first frustum. The hollow open ended first frustum can have a first frustum bottom and an opposing first frustum top. The first frustum bottom can have a first frustum bottom perimeter and the first frustum top can have a first frustum top perimeter. The first frustum bottom perimeter can be greater than the first frustum top perimeter. The first frustum top can be engaged or joined with the insertion member.

The applicator can comprise a seating member engaged with the insertion member. The first frustum top perimeter can be engaged with the insertion member through the seating member. The first frustum bottom can be oriented towards the insertion end of the insertion member. The first frustum bottom can be oriented away from the insertion end of the insertion member. The first frustum can be a conical frustum. The first frustum can have a curved slope.

The indicator member can have a first position and a second position. In the first position, the first frustum bottom can be oriented towards the insertion end of the insertion member. In the second position, the first frustum bottom can be oriented away from the insertion end of the insertion member.

The applicator can comprise a cushion member extending from at least a portion of the first frustum bottom perimeter of the first frustum. The first frustum can comprise at least one structural discontinuity. The first frustum can be comprised of a flexible material.

The applicator can be an applicator for an article selected from the group consisting of a tampon, a vaginal pessary, a rectal suppository, a vaginally administered medicine, and a vaginally administered contraceptive.

The indicator member can comprise a hollow open ended second frustum. The second frustum can have a second frustum bottom, a second frustum top, and a second frustum slope. The second frustum bottom can have a second frustum bottom perimeter and the second frustum top can have a second frustum top perimeter. The second frustum bottom perimeter can be greater than the second frustum top perimeter. The second frustum top perimeter can be equal to the first frustum bottom perimeter. The first frustum bottom perimeter can be engaged with the second frustum top perimeter. The first frustum can have a first frustum slope and the first frustum slope can differ from the second frustum slope.

The second frustum bottom can be oriented towards the insertion end of the insertion member. The second frustum bottom can be oriented away from the insertion end of the insertion member. The first frustum bottom can oriented towards the insertion end and the second frustum bottom can be oriented away from the insertion end of the insertion member. The first frustum bottom can be oriented away from the insertion end and the second frustum bottom can be oriented away from the insertion end of the insertion member. The second frustum can have at least one structural discontinuity.

The indicator member can be comprised of silicone. The applicator can be sized and dimensioned for insertion into a woman's vagina

### BRIEF DESCRIPTION OF THE DRAWINGS

FIG. 1 is an illustration of an embodiment of the applicator.

FIG. 2 is cross-sectional view of an embodiment of the applicator as indicated by Section 2-2.

FIG. 3 is an illustration of an indicator member transitioning from a first position to a second position.

FIG. 4 is an illustration of an embodiment of the applicator illustrating the spring behavior of an indicator member.

FIG. 5 is an illustration of an embodiment of the applicator.

FIG. 6 is an illustration of an embodiment of the applicator inserted into a cavity.

FIG. 7 is an illustration of an embodiment of the applicator inserted into a cavity.

FIG. 8 is an illustration of an embodiment of the applicator comprising a seating member.

FIG. 9 is an illustration of an embodiment of the applicator comprising a seating member.

FIG. 10 is an illustration of an embodiment of the indicator member.

FIG. 11 is an illustration of an embodiment of the applicator comprising a cushion member.

FIG. 12 is an illustration of an indicator member in which the cushion member is integral with the first frustum.

FIG. 13 is an illustration of an indicator member in which the cushion member is integral with the first frustum.

FIG. 14 is an illustration of an indicator member in which the cushion member extends radially outward from the first frustum and is biased towards the first frustum top.

FIG. 15 is cross-sectional view as indicated by Section 15-15.

FIG. 16 is an illustration of an indicator member in which the cushion member is not biased towards or away from the first frustum top.

FIG. 17 is a cross-sectional view as indicated by Section 17-17.

FIG. 18 is an illustration of an embodiment of an indicator member comprising structural discontinuities.

FIG. 19 is an illustration of an embodiment of an indicator member comprising a second frustum.

FIG. 20 is a cross-sectional view as indicated by Section 20-20.

FIG. 21 is an illustration of an embodiment of a second frustum transitioning from a first position to a second position.

FIG. 22 is an illustration of an embodiment of an applicator in which the second frustum bottom is oriented away from the insertion end of the insertion member.

FIG. 23 is an illustration of an embodiment of an applicator.

FIG. 24 is an illustration of an embodiment of the applicator inserted into a cavity.

FIG. 25 is an illustration of an embodiment of the applicator inserted into a cavity.

FIG. 26 is an illustration of an embodiment of the applicator inserted into a cavity.

FIG. 27 is an embodiment of the indicator member.

FIG. 28 is an illustration of an applicator having an indicator member shaped like the nipple from a baby bottle.

FIG. 29 is an embodiment of the applicator in which the indicator member partially circumscribes the periphery of the insertion member.

FIG. 30 is a cross section of an embodiment of a first frustum comprised of a stiff material rendered extensible by providing a plurality of corrugations and openable voids.

### DETAILED DESCRIPTION OF THE INVENTION

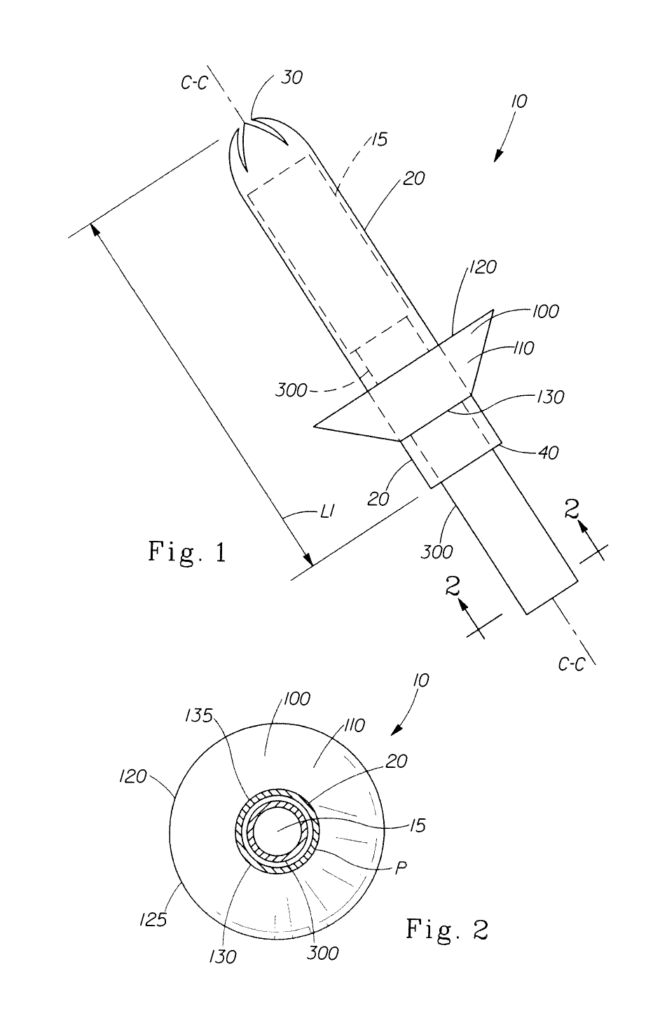

An illustration of one embodiment of the applicator 10 in profile is shown in FIG. 1. The applicator 10 comprises a hollow insertion member 20 and an indicator member 100. The insertion member 20 is adapted to house an object 15 to be inserted into a cavity. The applicator 10 has an insertion end 30 and an opposing gripper end 40. The insertion member 20 has a length LI. The length LI of the insertion member 20 is defined by the distance between the insertion end 30 and the gripper end 40. The applicator has a longitudinal centerline C-C. The longitudinal centerline C-C can be straight, curved, or a combination of straight portions and curved portions.

The length LI of the insertion member 20 can be between about 40 mm and about 100 mm. The length LI of the insertion member 20 can be sized and dimensioned to so that the applicator 10 can house a tampon or pessary having a length between about 30 mm and about 60 mm. The length LI of the insertion member 20 can be a function of the dimensions of the object contained therein and the depth to which the object is to be placed. The insertion member 20 can have an outer diameter between about 5 mm and about 30 mm. The insertion member 20 outer diameter can be between about 10 mm and about 21 mm. The inner diameter of the insertion member 20 can be between about 1 mm and about 29 mm. The inner diameter of the insertion member 20 can be between about 10 mm and about 19 mm. The inner diameter of the insertion member 20 can be between about 16 mm and about 19 mm. The insertion member 20 can be sized and dimensioned to be inserted into a woman's vagina. The dimensions are provided by way of example only and not to be limiting, as other dimensions can be appropriate depending on the task for which the applicator 10 is used. For instance, the outer diameter and length of an applicator sized and dimensioned to be used on a large mammal, such as an elephant, can be much larger than the particular dimensions disclosed herein.

In one embodiment, the indicator member 100 can comprise a hollow open ended first frustum 110. In another embodiment, the indicator member 100 can comprise part of a hollow open ended first frustum 110. The first frustum 110 has a first frustum bottom 120 and an opposing first frustum top 130.

A frustum is the part of a solid intersected by two planes. The solid can be a cone, a pyramid, a sphere, oblate spheroid, a prolate spheroid, an elliptic cone, an ellipsoid, a pseudosphere, a funnel, a hyperboloid, a lemon, a paraboloid, a pyramidal frustum, or any other geometric solid that is not a parallelepiped. The two planes can be parallel to one another or can be non-parallel to one another.

The applicator 10 can further comprise a plunger 300 slideably configured within the insertion member 20. In one embodiment, the cavity into which an object is to be placed is a woman's vagina.

As disclosed herein, the applicator 10 and object 15 are described primarily with respect to a tampon. However, it is to be understood that other devices, such as a pessary for treating urinary incontinence, a contraceptive device, a vaginally administered contraceptive, a vaginally administered medicine, a medical measurement device, a device for treating prolapse, or other device or article to be inserted into a woman's vagina may also be contained in the applicator 10 and discharged from the applicator 10. The object 15 can be a surgical instrument. The object 15 can be a measurement probe. The object 15 can be an object to be inserted in the body of an animal, can be an object to be inserted into a chamber, or can be an object to be inserted into a solid object into which an applicator 10 can be inserted.

The indicator member 100 can provide a shield that protects the user of the applicator 10 from contacting the body into which the object is to be inserted.

FIG. 2 is an end view, as indicated by Section 2-2, of one embodiment of the applicator 10 orthogonal to the longitudinal centerline C-C and viewed such that the gripper end 40 is oriented towards the viewer of FIG. 2. As shown in FIG. 2, the insertion member 20 has a periphery P. The shape defined by the periphery P is generally orthogonal to the longitudinal centerline C-C of the insertion member 20. The indicator member 100 circumscribes or partially circumscribes the periphery P of the insertion member 20. The first frustum 110 has a first frustum bottom 120. The first frustum bottom 120 has a first frustum bottom perimeter 125. The first frustum 110 also has a first frustum top 130. The first frustum top 130 has a first frustum top perimeter 135. The first frustum bottom perimeter 125 is greater than the first frustum top perimeter 135. The first frustum top 130 is engaged with the insertion member 20.

The terms circumscribe and circumscribing describe that the indicator member 100 is structured so as to be around or about the periphery P and is not limited to merely describing the relationship of an indicator member 100 around a circular insertion member 20. For instance, if the shape defined by the periphery P of the insertion member 20 is oval shaped, the indicator member 100 can circumscribe an oval shaped insertion member. Similarly, if the shape defined by the periphery P of the insertion member 20 is square, the indicator member 100 can circumscribe a square shaped insertion member. In essence, the term circumscribe is not limited to being around a circle. Partially circumscribing means partly around or partly about the periphery P.

The periphery P of the insertion member 20 can be circular. The periphery P of the insertion member 20 can be oval shaped. The periphery P of the insertion member 20 can be any closed geometric shape and an indicator member 100 that is around the insertion member 20 can be described as circumscribing the insertion member 20.

A first element is engaged with a second element if stress mobilized in the first element can be transmitted to the second element. The first element and the second element can be separated by one or more intermediate elements and still be engaged, so long as stress mobilized in the first element can be transmitted to the second element. A first element can be engaged with a second element by joining the two elements together. A first element can be considered to be engaged with a second element if the two elements are created from a continuous piece of material. An example in which a first element is engaged with a second element is a rubber band stretched about cylinder. Another example in which a first element is engaged with a second element can be created by the following steps: (1) taking a baby bottle nipple from which the tip is cut off and (2) forcing a cylinder having a diameter greater than the diameter of the opening on the tip of the nipple through the opening on the tip of the nipple. Another example in which a first element is engaged with a second element includes an arrangement in which the first element and second element are joined to one another. Elements can be joined to one another by an adhesive, fusion or compression bonding, mechanically attaching the elements, or other means for associating two elements with one another. These are only examples of structures in which first elements are engaged with second elements and are not all inclusive of the variety of means by which two elements can be engaged with one another.

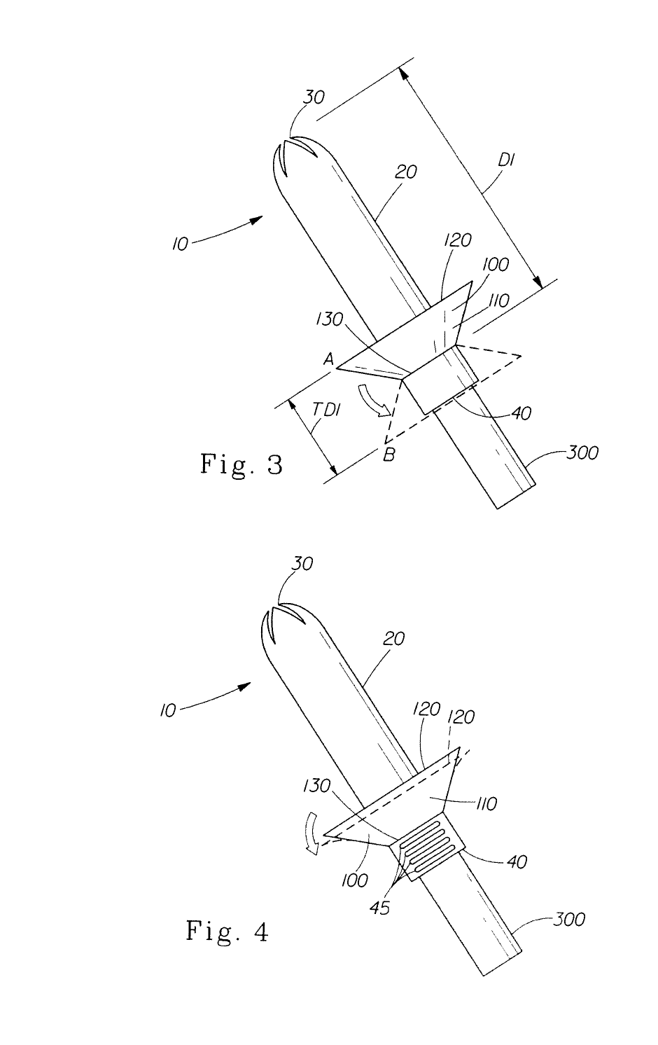

In one embodiment, the indicator member 100 provides a tactile signal that the insertion member 20 is properly inserted into the cavity into which an object is to be placed. The tactile signal can be provided by the indicator member 100 transitioning from a first position to a second position, as shown in FIG. 3. In the first position, the first frustum bottom 120 can be oriented towards the insertion end 30 of the insertion member 20 and the first frustum top 130 can be oriented away from the insertion end 30. In the second position, the first frustum bottom 120 can be oriented away from the insertion end 30 and the first frustum top 130 can be oriented towards the insertion end 30. The indicator member 100 can be at equilibrium and self sustaining in shape in the first position. That is, the indicator member 100 can be stable or static in the first position. The indicator member 100 can be stable or static and self sustaining in shape in the second position. The indicator member 100 can be unstable and not self sustaining in intermediate positions between the first position and the second position.

The hollow open ended first frustum 110 can be comprised of a flexible material. A flexible material is a material that does not significantly plastically deform, i.e. plastic deformation is less than 5%, when the hollow open ended first frustum transitions from a first equilibrium state to a second equilibrium state. A flexible material can have a modulus between about 3 MPa and about 30 MPa, by way of example and not to be limiting. A flexible material can have a durometer hardness between about 25 and about 45. A flexible material can have a durometer hardness between about 15 and about 70. Examples of flexible materials from which the hollow open ended first frustum 110 may be comprised include, but are not limited to, materials employed in nipples for human baby bottles. In general, the material for the hollow open ended first frustum 110 can have a sufficient stiffness such that the material, when employed in the structures disclosed herein, does not significantly deform under its own weight. In other words, the material can maintain its structural form under its own weight and is not overly floppy or droopy. The stiffness of a material is a function of the modulus of the material and the moment of inertia about the bending axis of interest. Thus, more stiff materials can be used provided that the moment of inertia about the bending axis of interest is reduced, for example by reducing the cross sectional area of the material. Similarly, less stiff materials can be used provided that the moment of inertia about the bending axis of interest is increased, for example by increasing the cross sectional area of the material. The hollow open ended first frustum 110 can be comprised of an elastic material. The hollow open ended first frustum 110 can be comprised of a deformable material. The terms elastic and deformable are taken to mean their ordinary engineering definitions within the scope of strains occurring as the first frustum 110 transitions from a first position to a second position.

The hollow open ended first frustum 10 can be comprised of materials stiffer than those above provided that the material is rendered to be extensible. An extensible material is a material that can be extended by at least 5% without tearing or altering the molecular structure of the material. Stiff materials can be rendered extensible by providing a plurality of corrugations. Corrugated materials can be extensible because the individual ridges and grooves can spread apart without significantly deforming the material between the ridges and grooves. Corrugations can have sharp folded edges or can be rounded folded edges. Stiff materials can also be rendered extensible by providing a number of voids in the material that are collapsed upon themselves and are openable. As the material is lengthened, the voids can open up and provide for expansion of the material. For instance, the hollow open ended first frustum 110 can be comprised of a cardboard. Cardboard can be rendered extensible by process known in the art for producing a plurality of ridges and grooves in the material including, but not limited to, corrugating and ring rolling. The hollow open ended first frustum 10 can be comprised of paper or laminated paper. The hollow open ended first frustum 10 can be comprised of stiff plastics rendered extensible as described above.

In one embodiment, the hollow open ended first frustum 1 10 can be rendered extensible by providing a plurality of corrugations and/or openable voids extending circumferentially and/or partially circumferentially about the longitudinal centerline of the first frustum 110, the longitudinal centerline of the first frustum 110 is generally coincident with the longitudinal centerline C-C of the applicator 10. In another embodiment, the hollow open ended first frustum I 10 can be rendered extensible by providing a plurality of corrugations and/or openable voids extending along the slope and/or partially along the slope of the first frustum 10, the slope of the first frustum 10 being the portion of the first frustum between the first frustum bottom 120 and the first frustum top 130. The first frustum 110 can be rendered extensible by a combination of a plurality of corrugations and/or openable voids extending circumferentially and/or partially circumferentially about the longitudinal centerline of the first frustum 110 and a plurality of corrugations and/or openable voids extending along the slope and/or partially along the slope of the first frustum 110. The first frustum 110 can also be rendered extensible by a plurality of corrugations and/or openable voids that are oriented at an angle relative to the longitudinal centerline of the first frustum 110. The first frustum 110 can be rendered extensible by a combination of corrugations and/or openable voids extending circumferentially and/or partially circumferentially about the longitudinal centerline of the first frustum 110 and a plurality of corrugations and/or openable voids extending along the slope and/or partially along the slope of the first frustum 110 and a plurality of corrugations and/or openable voids that are oriented at an angle relative to the longitudinal centerline of the first frustum 110.

A hollow open ended first frustum 110 fabricated with a material having a modulus between about 3 MPa and about 30 MPa and a thickness between about 0.5 mm and about 4 mm can have two or more equilibrium states. The ranges of modulus and thickness are provided by way of example and not to be limiting. In one embodiment, the first equilibrium state can be described as having an outside surface and an inside surface. By applying force to the hollow open ended first frustum 110, the hollow open ended first frustum 110 can be flipped inside out such that the outside surface in the first equilibrium state becomes the inside surface in the second equilibrium state and the inside surface in the first equilibrium state becomes the outside surface in the second equilibrium state. One can conceptualize the two equilibrium states being similar to a foot sock that is right side out being turned inside out such that the foot sock is wrong side out. Similarly, a hollow open ended first frustum 110 can be fabricated from a stiffer material that is rendered extensible.

For the first frustum 110 illustrated in FIG. 3, the first equilibrium position can be that illustrated in solid lines and the second equilibrium position can be that illustrated in dashed lines, with the arrows denoting movement of the first frustum bottom 120. The transition from the first position to the second position occurs rapidly once the first frustum 110 is stressed in the axial direction sufficiently to drive the transition. The transition of the first frustum 110 can provide a tactile sensation and the observer can feel a click, as mechanical energy stored in the first frustum 110 is rapidly released as a result of the transition.

For a tampon or pessary applicator, the force required to drive the transition from the first position to the second position can be between about 1 N and about 6 N. These forces are provided by way of example and not to be limiting.

The first frustum transition distance TD1, is defined as the distance by which the first frustum bottom 120 moves as the first frustum bottom 120 transitions from the first position to the second position. The transition distance TD1 can be about two times the distance that a user can effectively sense movement of the insertion member 20 as the insertion member 20 penetrates the cavity into which the object is to be placed. For an applicator designed for inserting objects into a woman's vagina, the first transition distance TD1 can be between about 10 mm and about 20 mm.

If the tactile signal is provided by the indicator member 100 transitioning from a first position to a second position, as shown in FIG. 3, the proper depth of insertion DI can be the distance between the insertion end 30 and the first frustum top 130.

A tampon or pessary can be positioned about 10 mm to about 40 mm above the hymenal ring, as measured from the base or trailing edge of the tampon or pessary. The desired depth of insertion DI can depend on the force applied to the applicator 10 as the user inserts the applicator 10 into a cavity. If the indicator member 100 is designed to transition from a first position to a second position, as shown in FIG. 3, and the signal being the sensation of the first frustum top 130 butting up against the hymenal ring, the depth of insertion DI can be about 10 mm to about 40 mm.

The first frustum 110 can be comprised of a material selected from the group consisting of rubber, NEOPRENE®, low density polyethylene, silicone, NALGENE®, and foam. Other soft, pliable, and flexible materials are contemplated. The force required drive the transition of the first frustum 110 from the first position to the second position and the transition distance TD1 can be design parameters and can be factored into design by selecting the type of material or materials used to construct the indicator member 100, selecting the geometry of the indicator member 100, and selecting the structure of the first frustum 110.

In another embodiment, as the user of the applicator 10 inserts the insertion member 20 into a cavity into which an object is to be placed, the tactile signal to the user that that insertion member 20 is inserted into the cavity to the proper depth can be generated by the first frustum bottom 120 coming into contact with the surface surrounding the cavity. In this embodiment, the first frustum bottom 120 can be oriented towards the insertion end 30 of the insertion member 20. For instance, if the applicator is used by a woman to place a tampon in her vagina, the tactile sensation can be the result of the first frustum bottom 120 coming into contact with her labia, skin surrounding her labia, and/or tissue surrounding her hymenal ring. A woman may sense the first frustum bottom 120 touching her labia, skin surrounding her labia, and/or tissue surrounding her hymenal ring as sensed though nerve receptors in these body parts or through nerve receptors in her hand that is holding the applicator 10.

Without being bound by theory, it is thought that a first frustum 110, as disclosed herein, can behave as a mechanical spring if the insertion member 20 is inserted into the cavity to a depth greater than intended. As the insertion member 20 is pushed into the cavity, portions of the first frustum bottom perimeter 125 can come into contact with material, which can be tissue, surrounding the cavity and provide resistance against deeper insertion into the cavity. As an increased amount of force is applied to the insertion member 20, which might occur by the user pushing on the insertion member 20, gripper end 40, or plunger 300, some of the force can be transmitted through the first frustum 110 to the first frustum bottom perimeter 125. In response to the force, the first frustum bottom perimeter 125 can be lengthened or stretched and the first frustum bottom perimeter 125 can flare out or crumple, thereby mobilizing some of the force applied to the insertion member 20, as shown in FIG. 4. If the force applied is great enough, the first frustum 110 can transition from a first position to a second position as shown in FIG. 3 and discussed above.

The first frustum 110 can provide a cushion to distribute the force applied to the insertion member 20 as the insertion member 20 is pushed into the cavity. If the applicator is used to insert an object into a woman's vagina, the force applied to the insertion member 20 can be distributed to the labia and surrounding skin, making the process of inserting the insertion member 20 more comfortable, and the user can sense the proper depth of insertion as the first frustum bottom 120 contacts her labia, skin surrounding her labia, and/or tissue surrounding her hymenal ring.

The gripper end 40 of insertion member 20 can comprise a finger grip 45, as shown in FIG. 4. The first frustum top 130 can be near the finger grip 45. The finger grip 45 allows the consumer to more securely hold the applicator 10 while inserting the insertion member 20 and expelling the object contained in the insertion member 20, and withdrawing the insertion member 20. A variety of finger grip 45 designs are known in the art. For example, a finger grip 45 can be formed by embossing the outside surface of the insertion member 20. The embossed portion of the insertion member 20 can be in the form of a series of circumferential rings or a series of discrete raised or depressed dots. The finger grip 45 can be formed as part of an injection molding process. Examples of a finger grip 45 can be found in U.S. Pat. No. 6,045,526, U.S. Pat. No. 4,573,963, U.S. Pat. No. 4,412,833, and U.S. Pat. No. 6,685,787.

The plunger 300 of the applicator 10 of the present invention can also have a plunger grip portion or plunger gripping means. The plunger grip on the plunger 300 can be any type of embossment or depression known in the art. Examples of plunger grips and plungers 300 can be found in U.S. Pat. No. 3,068,867, European Patent Application publication number 034922 published by Alexander, et al. on Mar. 1, 1990, U.S. Pat. No. 3,196,873, and U.S. Pat. No. 3,068,867. The plunger 300 can have a length between about 45 mm to about 100 mm, as measured parallel to the longitudinal centerline of the applicator 10.

The plunger 300 can have a stopping mechanism to keep the plunger 300 from being plunged too far. Examples of stopping mechanisms are disclosed in U. S. Pat. No. 6,019,744 and U.S. Pat. No. 6,450,986.

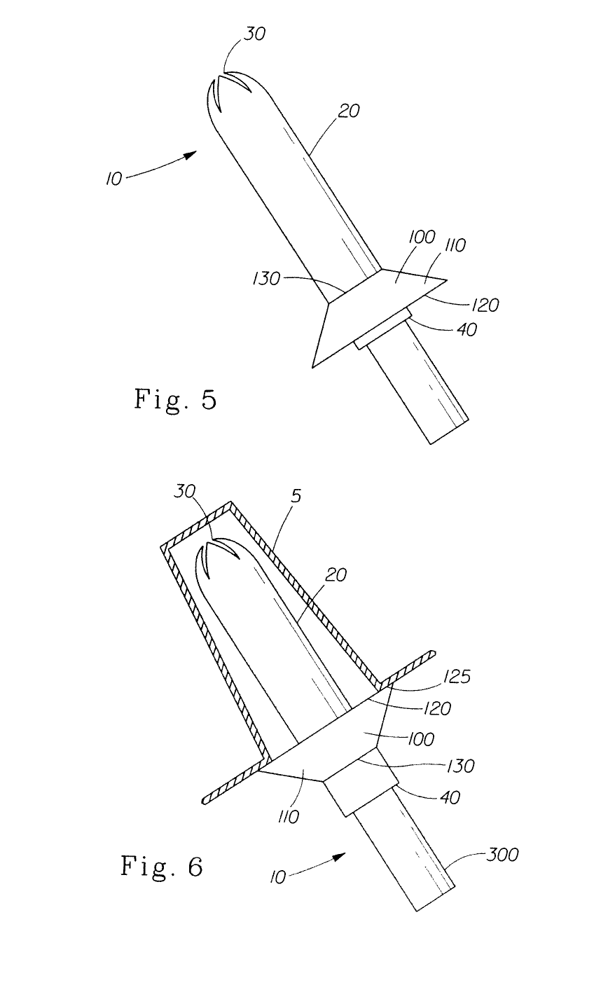

If the first frustum bottom 120 is oriented away from the insertion end 30 of the insertion member 20, as shown in FIG. 5, the tactile signal can be caused by portions of the first frustum 110 near the first frustum top 130 coming into contact with the surface surrounding the cavity as the user inserts the insertion end 30 into the cavity. For instance, if the applicator is used by a woman to place a tampon in her vagina, the tactile sensation can be the result of portions of the first frustum 110 near the first frustum top 130 coming into contact with her labia, skin surrounding her labia, and or tissue surrounding her hymenal ring. The woman may sense portions of the first frustum 110 near the first frustum top 130 touching her body through nerve receptors located near where contact occurs or through nerve receptors in her hand holding the applicator 10. The first frustum 110, oriented as shown in FIG. 5, can act as a cushion against the force applied to the insertion member 20. Without being bound by theory, it is thought that as the first frustum 110 contacts the labia and surrounding skin, the tendency is for the first frustum 110 to spread apart the labia. When sufficient force developed in the first frustum 110, the first frustum 110 can compress, deform, and/or collapse, thereby providing a cushion. The first frustum 110 oriented as shown in FIG. 5 can function as a shield that keeps the fingers of the person using the applicator from touching the cavity or portions surrounding the cavity into which the insertion member 20 is inserted.

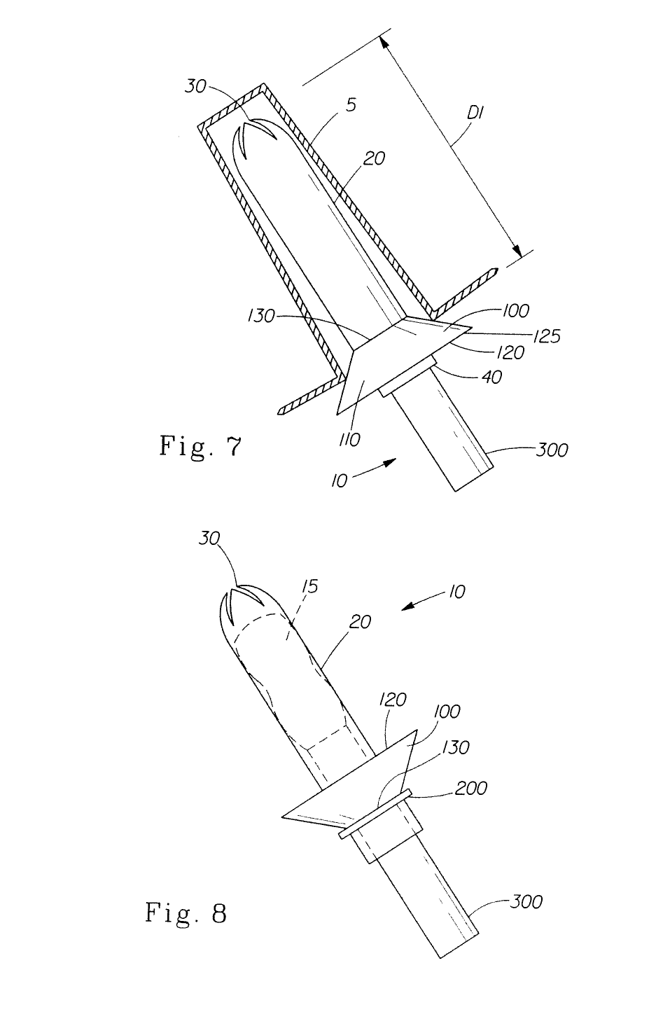

FIGS. 6 and 7 illustrate how one embodiment of the indicator member 100 can behave as the insertion member 20 penetrates a cavity 5. In a first position, shown in FIG. 6, the insertion member 20 can be partially inserted into the cavity 5. The first frustum bottom 120 can contact the boundaries of cavity 5 and can provide a tactile signal that the insertion member 20 is inserted to a desired depth, as discussed above. Further penetration of the insertion member 20 into the cavity 5 can result in the indicator member 100 transitioning from a first position, in which the first frustum bottom is oriented towards the insertion end 30 and the first frustum top 130 is oriented away from the insertion end 30 as shown in FIG. 6, to a second position, in which the first frustum top 130 is oriented towards the insertion end 30 and first frustum bottom 120 is oriented away from the insertion end 30, as shown in FIG. 7. The wedge formed by the indicator member 100 can resist further penetration of the insertion member 20 into the cavity 5. The indicator member can be sized and configured to permit the insertion member 20 to penetrate into the cavity 5 to the desired depth depending on the size and shape of the opening of the cavity and the size and shape of the indicator member 100.

As illustrated in FIG. 7, if the indicator member 100 partially penetrates the cavity 5, the depth of insertion DI can be measured from the insertion end 30 to the location at which the indicator member 100 contacts the boundary of the cavity 5. If the cavity 5 is a woman's vagina, the tissues surrounding the hymenal ring are soft and when pressure is applied to the surrounding tissues, the tissues compress. The proper depth of insertion DI can be provided by considering the amount of force the user will apply to insert the applicator 10 into the cavity 5 and the deformation of tissues or structures surrounding the cavity 5 resulting from the indicator member 100 being forced against the boundaries of the cavity 5. Furthermore, the indicator member 100 may deform under pressure and the amount of deformation of the indicator member 100 can be factored into designing for the proper depth of insertion. The proper depth of insertion for other cavities having soft boundaries can be provided for similarly.

The insertion member 20 and plunger 300 can be formed from a spirally wound, convolutedly wound, or longitudinally seamed hollow tube. Suitable materials for the insertion member 20 and plunger 300 include, but are not limited to, paper, paperboard, cardboard, or a combination thereof. The insertion member 20 and plunger 300 can also be injection molded, extruded, or formed from flexible plastic, such as thermoformed from a plastic sheet or folded or wound from plastic film. The insertion member 20 and plunger 300 can also be formed from a combination of paper and plastic. The insertion member 20 and plunger 300 can be constructed from a laminate comprising two or more different materials in a layered arrangement. When two or more layers are used, the layers can be spirally wound, convolutedly wound, or longitudinally seamed to form an elongated cylinder.

The dimensions of the insertion member 20 and plunger 300 can be dictated by the size of the object to be placed in the cavity, the size of the cavity into which the object is being placed, the size of the opening of the cavity, and the size of the applicator 10 that can be easily handled by the person or machine inserting the object. The insertion member 20 and plunger 300 may be sized to accommodate a tampon used by women to manage vaginal discharge and menstruation. There are a variety of sizes of tampons. The size of a tampon can depend on the absorbency. For larger mammals, such as farm animals and zoo animals, a larger tampon might be used, thereby requiring the maximum dimension of the insertion member 20, as measured orthogonal to the longitudinal centerline C-C, to be larger than about 19 mm.

The shape of a cross section of the insertion member 20 cut orthogonal to the longitudinal centerline C-C can be any shape desired. For instance, for a tampon used by women, the shape of the cross section of the insertion member 20 cut orthogonal to the longitudinal centerline C-C can be selected from the group consisting of a circle, an oval, a curved shape, an irregular curved shape, a polygon, and a shape having a combination of curved and straight edges. The shape of a cross section of the insertion member 20 cut orthogonal to the longitudinal centerline C-C can be a shape that approximately conforms to the shape of the tampon, or other object, enclosed within the insertion member 20. The shape of a cross section of the insertion member 20 cut orthogonal to the longitudinal centerline C-C can be a shape that approximately conforms to the shape of the cavity or opening of the cavity.

In general, the size and shape of a cross section of the insertion member 20 cut orthogonal to the longitudinal centerline C-C should not conform too closely with the shape of the object contained therein so that the object can easily be ejected by applying the desired force to the plunger 300.

The plunger can be shaped such that the cross section of the plunger 300 cut orthogonal to the longitudinal centerline C-C fits within the cross section of the insertion member 20 cut orthogonal to the longitudinal centerline C-C. For an insertion member 20 having a circular cross section orthogonal to the longitudinal centerline C-C, the plunger 300 can also have a circular cross section orthogonal to the longitudinal centerline C-C and a diameter less than or about equal to the inside diameter of the insertion member 20. The plunger 300 can be sized and dimensioned to be able to slide within the insertion member 20. For applicators 10 used to place tampons in women, the plunger 300 can be sized and dimensioned to have only minimal clearance between the plunger 300 and the interior wall of the insertion member.

The thickness of the material forming the insertion member 20 can be about 0.1 mm to about 2 mm. The thickness of the material forming the insertion member 20 can be greater than about 2 mm.

The insertion end 30 of the insertion member 20 can be open-ended, closed-ended, or partially open-ended. In one embodiment, the insertion end 30 of the insertion member 20 can be closed-ended and slit in a pattern comprising petals. If a structure comprising petals is used, as the object is expelled from the insertion member 20 by the plunger 300, the petals open, thereby allowing the object to pass through the insertion end 30. In other embodiments, the insertion end 30 of the insertion member 20 can comprise a pleated structure, a corrugated structure, or cap. If a cap is used, the cap can be a film cap that is punctured as the object contained within the insertion member 20 is expelled, with the cap remaining attached to the insertion member to prevent the cap from being accidentally placed in the cavity into which the object is being place.

In one embodiment, the applicator 10 can further comprise a seating member 200, an example of which is shown in FIG. 8. The seating member 200 can be engaged with the insertion member 20. The first frustum top 130 can be engaged with the insertion member 20 through the seating member 200. The seating member 200 can be an annulus of material engaged with the insertion member 20, as shown in FIG. 8.

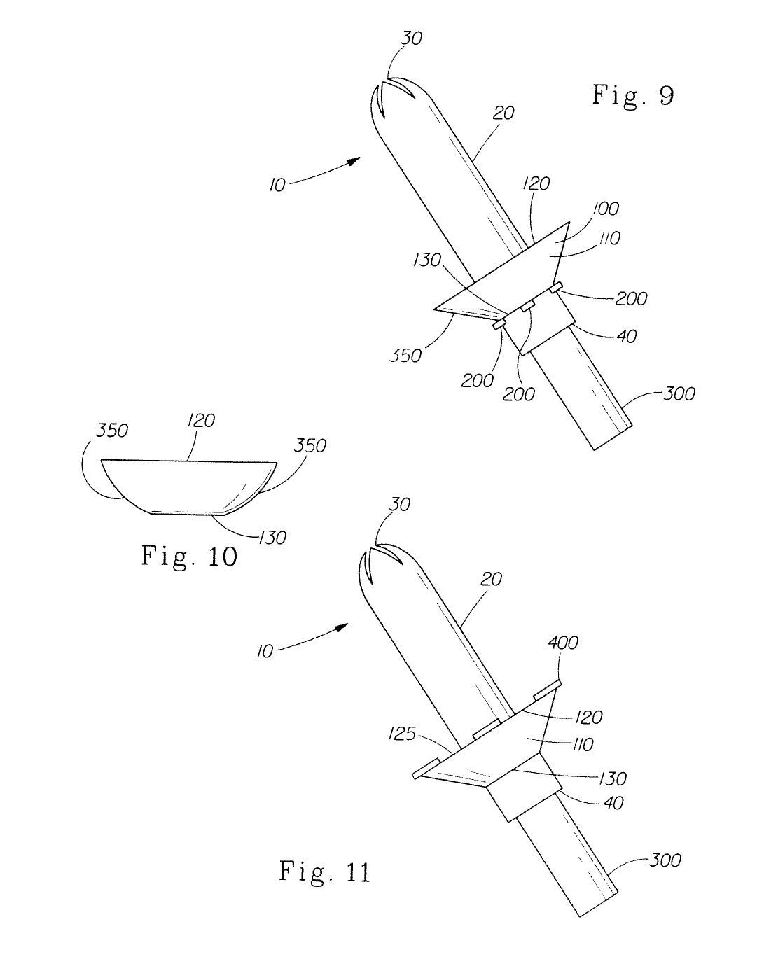

The seating member 200 can be a portion or portions of an annulus of material engaged with the insertion member 20, as shown in FIG. 9. The seating member 200 can be small raised portions extending from the insertion member 20. The seating member 200 can be integral with the insertion member 20 in that the insertion member 20 and seating member 200 are formed in a single process. The seating member 200 can be fixed in position by bonding the seating member 200 to the insertion member 20. The seating member 200 can be bonded to the insertion member 20 by adhesive bonding, thermal bonding, ultrasonic bonding, compression bonding, or any other method for bonding to like materials or dissimilar materials.

The first frustum 110 can be a conical frustum. A conical frustum has the shape of cone from which the pointed top is removed. The top of the cone can be removed such that the base of the frustum and top of the frustum are parallel to one another or non-parallel to one another. A hollow open ended first frustum 110, wherein the first frustum 110 is a conical frustum is illustrated, for example, in FIGS. 1, 8, and 9. The first frustum slope 350 is the shape of the shortest line, which can be curved or straight, drawn on the surface of the first frustum 110 from a point on the first frustum bottom 120 connecting the first frustum bottom 120 and the first frustum top 130. The first frustum slope 350 for a conical frustum is a straight line. The first frustum slope 350 can be described as the portion of the first frustum 110 that is between the first frustum bottom 120 and the first frustum top 130.

The first frustum slope 350 can be quantified against the longitudinal centerline C-C of the insertion member on which the indicator member 100 is disposed. The slope of a frustum can be quantified using the common approach for quantifying slopes of lines or curves as “rise over run” or deviation of a portion of a line or curve as compared to a straight reference line with the slope computed based on the top and bottom edges of the frustum.

The first frustum 10 can be selected from the group consisting of a spherical frustum, part of an oblate spheroid, part of a prolate spheroid, part of an elliptic cone, part of an ellipsoid, part of a pseudosphere, part of a funnel, part of a hyperboloid, part of a lemon, part of a paraboloid, and a pyramidal frustum.

The first frustum 110 can be selected from the group consisting of a radial part of a spherical frustum, a radial part of an oblate spheroid, a radial part of a prolate spheroid, a radial part of an elliptic cone, a radial part of an ellipsoid, a radial part of a pseudosphere, a radial part of a funnel, a radial part of a hyperboloid, a radial part of a lemon, a radial part of a paraboloid, and a radial part of a pyramidal frustum.

A cross section of a hollow open ended first frustum 110 wherein the first frustum 110 is part of a sphere is illustrated in FIG. 10. The first frustum slope 350 is a curved line for a first frustum 10 wherein the first frustum 10 is part of a sphere, part of an oblate spheroid, part of a prolate spheroid, part of an elliptic cone, part of an ellipsoid, part of a pseudosphere, part of a funnel, part of a hyperboloid, part of a lemon, and part of a paraboloid.

A pyramidal frustum is a frustum made by slicing off the top of a pyramid. A pyramid is a polyhedron with one face a polygon, known as the base, and all the other faces are triangles meeting at a common polygon vertex, called the apex. The first frustum slope 350 for a first frustum 110 wherein the first frustum 110 is a pyramidal frustum is a straight line. The first frustum slope 350 for a first frustum 110 wherein the first frustum 110 is part of a funnel is a curved line.

The first frustum slope 350 can be a curved shape corresponding to a defined geometric shape recognized by a person skilled in the art of geometry or can have a curved shape that does not correspond to a classically defined geometric shape. That is, the first frustum 10 can have a shape other than those defined by classical geometric terms.

The first frustum slope 350 can have an irregular shape that is a combination of curved and straight segments, a combination of curved segments having different curvature, or a combination straight segments that are not parallel to adjacent segments. For instance, the first frustum slope 350 can have the shape of a nipple for a baby bottle. Nipples for baby bottles can have a broadly curving base that reverses upon itself more steeply towards the tip of the nipple from which fluid is dispensed.

The first frustum bottom 120 can have a perimeter that is irregularly shaped, i.e. not circular. For example, the first frustum bottom 120 can have a wavy shape in which different portions of the perimeter of the first frustum bottom 120 are further away from the insertion member 20 than other portions of the perimeter of the first frustum bottom 120.

An embodiment in which the applicator 10 further comprises one or more cushion members 400 extending from at least a portion of the first frustum bottom perimeter 125 of the first frustum 110 is shown in FIG. 11. The cushion member 400 can extend about the entire first frustum bottom perimeter 125. The cushion member 400 can be comprised of a material that feels softer and/or more pliable than the first frustum 110. The cushion member 400 can be comprised of a polymeric material including, but not limited to, NEOPRENE®, polyethylene, low density polyethylene, rubber, silicone, and NALGENE®. The cushion member 400 can be comprised of a foam, such as a foam of polyester, a foam of polyurethane, or other similar foam. The cushion member 400 can be comprised of a woven or nonwoven material. The cushion member 400 can be comprised of a material that has a modulus lower than that of the material used to form the first frustum 110. The cushion member 400 can be joined to the first frustum bottom perimeter. The cushion member 400 can be comprised of the same material as the first frustum 110.

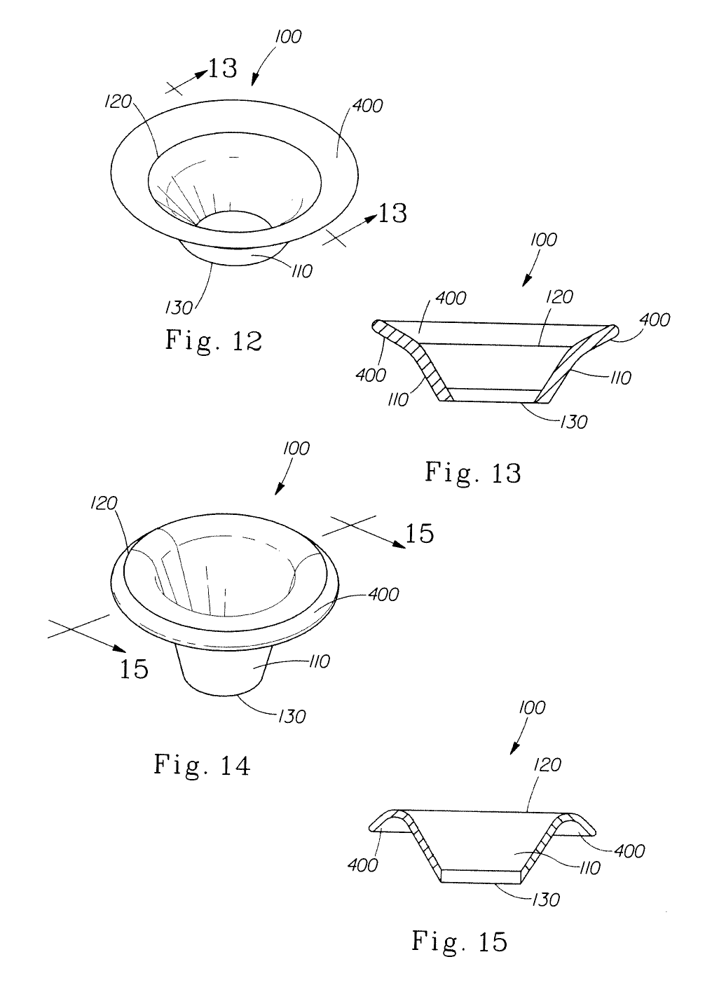

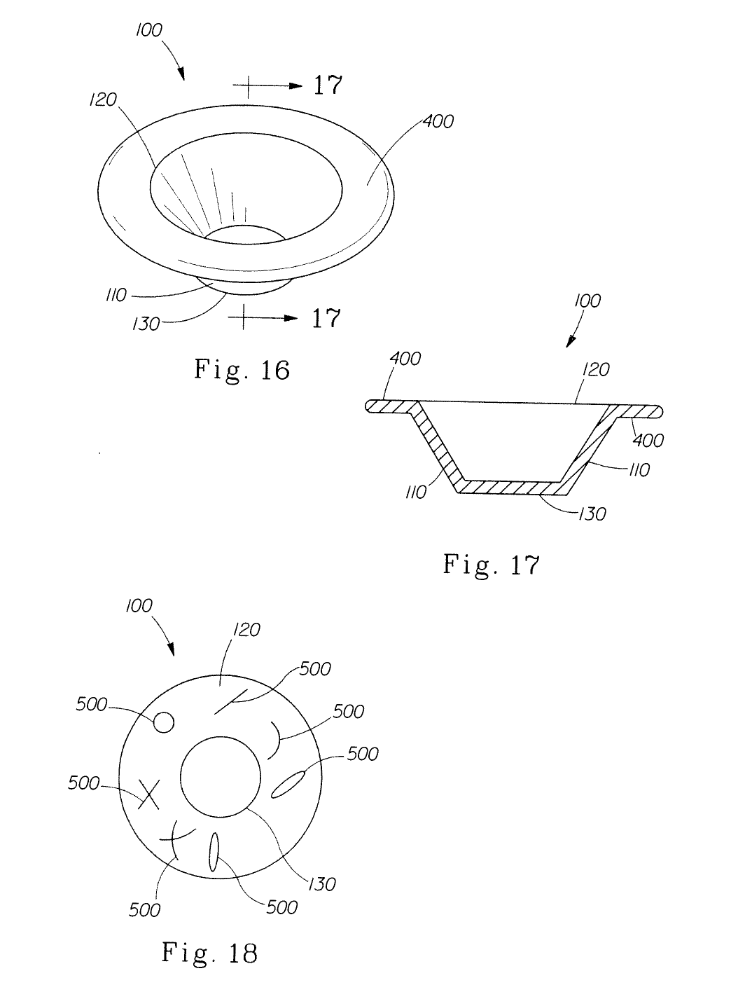

In one embodiment of the indicator member 100, an example of which is shown in FIGS. 12 and 13, the cushion member 400 can be integral with the first frustum 110 in that cushion member 400 and first frustum 110 are formed in a single process and are integral with one another without the need to bond the cushion member 400 and the first frustum 110. As shown in FIGS. 12 and 13, the cushion member 400 can extend radially outward from the first frustum 110. The cushion member 400 can extend radially outward from the first frustum 110 and be biased in a direction oriented away from the first frustum top 130, as shown in FIGS. 12 and 13. The cushion member 400 can extend radially outward from the first frustum 110 and be biased towards the first frustum top 130 as shown in FIGS. 14 and 15. The cushion member 400 need not be biased towards the first frustum top 130 or away from the first frustum top 130, as shown in FIGS. 16 and 17.

In one embodiment, an example of which is shown in FIG. 18, the indicator member 100 and/or cushion member 400 can have one or more structural discontinuities 500. A structural discontinuity 500 can be a portion of the indicator member 100 and/or cushion member 400 in which the mechanical properties or mechanical behavior of that portion of indicator member 100 and/or cushion member 400 differs from portions of the indicator member 100 and/or cushion member 400 adjacent to the structural discontinuity 500. The structural discontinuity 500 can be selected from the group consisting of a slit, intersecting slits, a slot, a perforation, zones of varied modulus, and an aperture. A slit is a structural discontinuity 500 in which when the structural discontinuity 500 is in a relaxed state, there is not an open space. A slot is a structural discontinuity 500 in which when the structural discontinuity 500 is in a relaxed state there is an opening having an aspect ratio greater than about 1. A zone of varied modulus is a zone in which the modulus of the material in one region differs from the modulus of the material in adjacent regions. A zone of varied modulus may be formed by altering the geometric shape of the material such that the material is corrugated, embossed, scored, or otherwise structurally altered. A zone of varied modulus may be formed by altering the molecular structure of the material by heating, cooling, stretching, compressing, or other wise imparting energy to the material. An aperture is a structural discontinuity 500 in which when the structural discontinuity 500 is in a relaxed state there is an opening having an aspect ratio of about 1. Slits, intersecting slits, and slots can be curved structures.

A structural discontinuity 500 can also be a region of the indicator member 100 in which the molecular structure of the region of the indicator member having a structural discontinuity 500 is different from the molecular structure of an adjacent region not having a structural discontinuity. The molecular structure of a portion of the indicator member can be altered by using a different material to fabricate the structural discontinuity. The molecular structure of a portion of the indicator member can also be altered by applying stress to a portion of the indicator member beyond the yield point of the material comprising the indicator member 100 which can cause a reorientation of the molecular structure of the material as well as other techniques known in the art.

A structural discontinuity 500 can also be a region or portion of the indicator member 100 having a thickness that is different from the thickness of adjacent regions or portions of the indicator member 100, which results in a change in the bending behavior of the indicator member 100.

In one embodiment, the indicator member 100 is reusable. An indicator member 100 that is reusable can be removed from one applicator 10 and used on another applicator 10. For example, consumer products companies sometimes package tampons in a package having more than one applicator 10, each applicator 10 containing a single tampon. An indicator member 100 that is reusable can be in the possession of the user of the tampon prior to purchase or can be packaged with the tampons. The user can transfer the indicator member 100 from one applicator 10 to another applicator 10 as she uses the tampon enclosed within each applicator 10. A reusable indicator member 100 can make packaging multiple tampons more convenient and economical for the user.

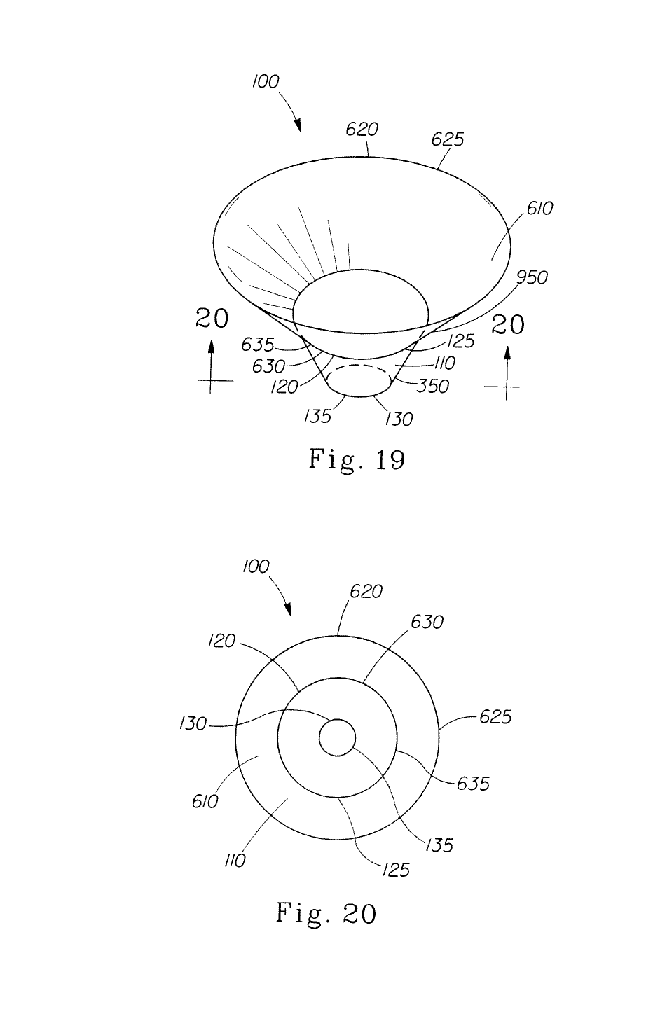

In another embodiment, an example of which is shown in FIGS. 19 and 20, the indicator member can comprise a hollow open ended second frustum 610 having a second frustum bottom 620, a second frustum top 630 and a second frustum slope 950. The second frustum bottom 620 has a second frustum bottom perimeter 625. The second frustum top 630 has a second frustum top perimeter 635. The second frustum bottom perimeter 625 can be greater than the second frustum top perimeter 635. The second frustum top perimeter 635 can be equal to the first frustum bottom perimeter 125. The first frustum bottom perimeter 125 can be engaged with the second frustum top perimeter 635. The first frustum slope 350 can differ from the second frustum slope 950.

The second frustum slope 950 is the shape of the shortest line drawn on the surface of the second frustum 610 from a point on the second frustum bottom 620 connecting the second frustum bottom 620 and the second frustum top 630. The first frustum slope 350 can be greater than the second frustum slope 950. The first frustum slope 350 can be less than the second frustum slope 950. The second frustum slope can be quantified as described previously in regard to the first frustum slope 350.

The second frustum 610 can be comprised of the same materials as the first frustum 110. The second frustum 610 can have a thickness between about 0.5 mm and about 4 mm.

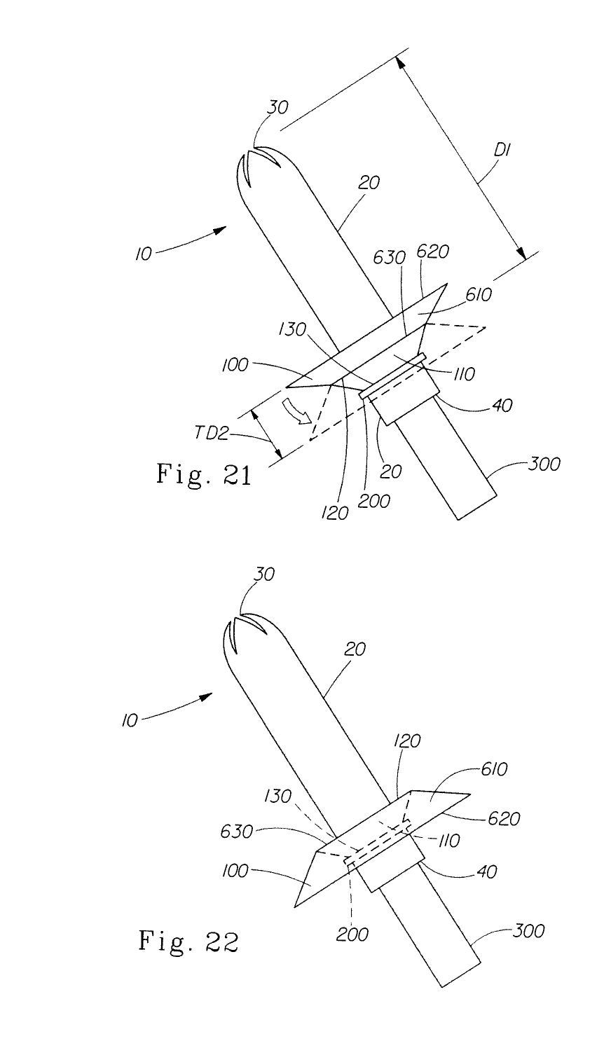

The embodiment for the indicator member 100 illustrated in FIGS. 19 and 20 can have three positions. A first position is illustrated in FIG. 21. In the first position, the second frustum bottom 620 can be oriented towards the insertion end 30, the second frustum top 630 can be oriented away from the insertion end 30, the first frustum bottom 120 can be oriented towards the insertion end 30, and the first frustum top 130 can be oriented away from the insertion end 30. In FIG. 21, the dashed lines illustrate a second position for the indicator member 100. The indicator member can be transitioned from the first position to the second position by force applied to the second frustum bottom 620 as a result of the applicator 10 being forced into a cavity.

In the second position, the second frustum bottom 620 can be oriented away from the insertion end 30, the second frustum top 630 can be oriented towards the insertion end 30, the first frustum bottom 120 can be oriented towards the insertion end 30, and the first frustum top 130 can be oriented away from the insertion end 30, as is shown in FIG. 22. The second frustum transition distance TD2 is defined as the distance by which the second frustum bottom 620 moves as the indicator member 100 transitions from a first position to a second position, as shown in FIG. 21. FIG. 22 also illustrates the second position for the indicator member 100.

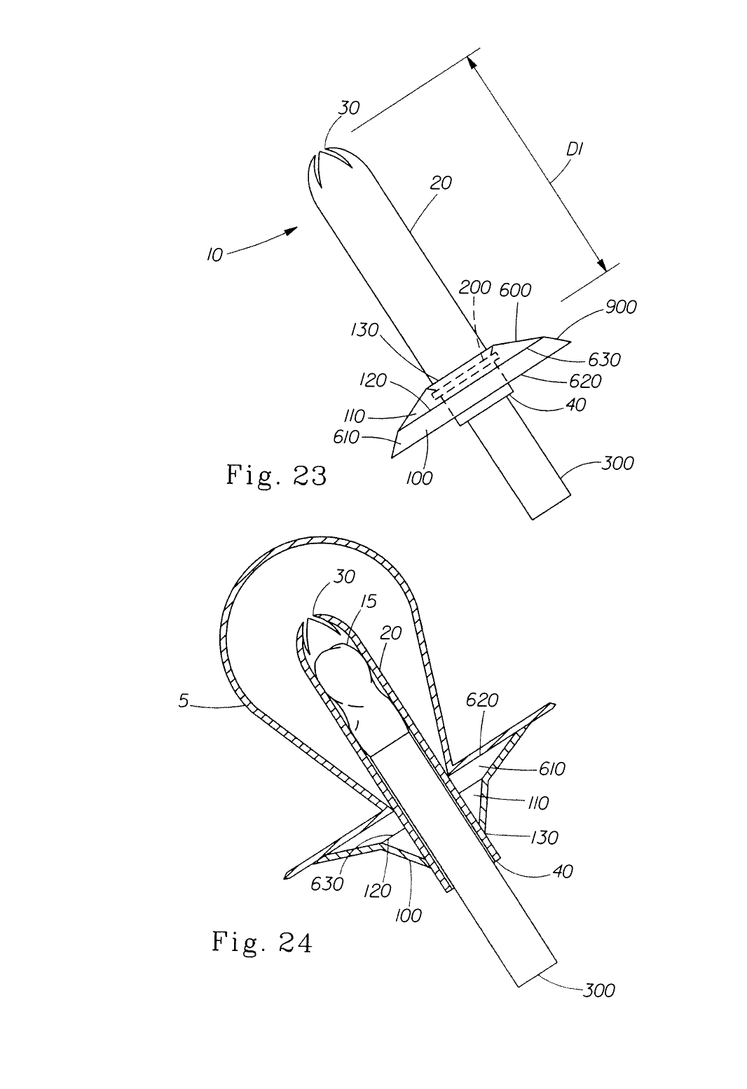

In a third position, an embodiment of which is illustrated in FIG. 23, the second frustum bottom 620 can be oriented away from the insertion end 30, the second frustum top 630 can be oriented towards the insertion end 30, the first frustum bottom 120 can be oriented away from the insertion end 30, and the first frustum top 130 can be oriented towards the insertion end 30.

The indicator member 100 can be stable or static and self sustaining in shape in the first position, second position, and third position. The indicator member 100 can be unstable and not self sustaining in intermediate positions between the first position and the second position and in intermediate positions between the second position and the third position.

In one embodiment, an indicator member 100 having three positions provides a tactile signal that the insertion member 20 is properly inserted into the cavity into which an object is to be placed. For the indicator member 100 having three positions, the tactile signal can be provided by the indicator member 100 transitioning from a first position to a second position, as shown in FIG. 21. In the first position, the second frustum bottom 620 can be oriented towards the insertion end 30 of the insertion member 20. In the second position, the second frustum bottom 620 can be oriented away from the insertion end 30 of the insertion member 20. The force required to drive the transition from the first position to the second position can be between about 1 N and about 6 N. The force required to drive the transition from the first position to the second position can be between about 0.1 N and about 20 N. These forces are provided by way of example and not to be limiting. The force required drive the transition from the first position to the second position and the distance the applicator 10 moves along the longitudinal centerline C-C as the transition between the first position and second position can be design parameters and can be controlled in design by selecting the type of material or materials used to construct the indicator member 100, selecting the geometry of the indicator member 100, and selecting the structure of the indicator member 100.

The second frustum transition distance TD2 can be between about 10 mm and about 20 mm. These distances are provide by way of example and not to be limiting. The second frustum transition distance TD2 can be about two times the distance that a user can effectively sense movement of the insertion member 20 as the insertion member 20 penetrates the cavity into which the object is to be placed.

If the tactile signal is provided by an indicator member 100 having three positions transitioning from a first position to a second position, as shown in FIG. 21, the proper depth of insertion DI can be the distance between the insertion end 30 and the second frustum top 630.

In one embodiment of the applicator 10, the user of the applicator can start with the applicator 10 in a position shown in FIG. 22. In this position, the portions of the indicator member near the first frustum bottom 120 and second frustum top 630 provide a springy cushion and the user's gripping fingers can be shielded from the cavity, such as a vagina, by the indicator member 100. As the user inserts the applicator 10 further into the cavity, the first frustum bottom 120 transitions from being oriented towards the insertion end 30, as shown in FIG. 22, to being oriented away from the insertion end 30, as shown in FIG. 23. The user can feel the transition. For a tampon applicator, as the transition occurs, the applicator 10 can be further pushed into the vagina until the first frustum top 130 contacts the hymenal ring or tissue surrounding the hymenal ring. A tactile signal to the user can be sensed as the first frustum top 130 contacts the hymenal ring or surrounding tissue. The indicator member 100 can also form a shield between the user's gripping fingers and the cavity. If the applicator 10 is for inserting a tampon into a vagina, the depth of insertion DI can be set based on the location of the first frustum top 130 being in contact with the woman's hymenal ring, such that the depth of insertion DI is between about 5 mm to about 10 mm or between about 5 mm and 30 mm.

If the second frustum bottom 620 is oriented towards the insertion end 30 of the insertion member 20, the tactile signal can be caused by the second frustum bottom 620 coming into contact with the surface surrounding the cavity. For instance, if the applicator is used by a woman to place a tampon in her vagina, the tactile sensation can be the result of the second frustum bottom 620 coming into contact with her labia, skin surrounding her labia, and/or tissue surrounding her hymenal ring.

Without being bound by theory, it is thought that a second frustum 610 comprised of a material having a modulus between about 3 MPa and 30 MPa and a thickness between about 0.5 mm and about 4 mm can behave like a mechanical spring if the insertion member 20 is inserted into a cavity, such as a vagina, to a depth greater than desired. The ranges in modulus and thickness are provided by way of example and not to be limiting. As the insertion member 20 is pushed into the cavity, portions of the second frustum bottom perimeter 625 can come into contact with material surrounding the cavity and provide resistance against deeper insertion into the cavity. As an increased amount of force is applied to the insertion member 20, some of the force is transmitted through the second frustum 610 to the second frustum bottom perimeter 625. In response to the force, the second frustum bottom perimeter 125 can be lengthened or stretched and the second frustum bottom perimeter 625 can flare out, thereby mobilizing some of the force applied to the insertion member 20. If the force applied is great enough, the second frustum 610 can transition from a first position to a second position as shown in FIG. 21.

The second frustum 610 can provide a cushion to distribute the force applied to the insertion member 20 as the insertion member 20 is pushed into the vagina. The force applied to the insertion member 20 can be distributed to the labia, surrounding skin, or tissue surrounding the hymenal ring making the process of inserting the insertion member 20 more comfortable and the user can sense the proper depth of insertion as the second frustum bottom 620 comes into contact with her labia, skin surrounding her labia, and/or tissue surrounding her hymenal ring. The woman may sense the second frustum bottom 620 coming into contact with her labia, skin surrounding her labia, and/or tissue surrounding her hymenal ring as sensed through nerve receptors in her hand gripping the gripper end 40 of the insertion member 20 as she inserts the insertion member 20 into her vagina.

If the second frustum bottom 620 is oriented away from the insertion end 30 of the insertion member 20, as shown in FIG. 22, the tactile signal can be caused by portions of the second frustum 610 near the second frustum top 630 or portions of the first frustum bottom 120 near the second frustum top 630 coming into contact with the surface surrounding the cavity. For instance, if the applicator is used by a woman to place a tampon or pessary in her vagina, the tactile sensation can be the result of portions of the second frustum 610 near the second frustum top 630 or portions of the first frustum bottom 120 near the second frustum top 630 coming into contact with her labia and/or skin surrounding her labia. The woman may sense portions of the second frustum 610 near the second frustum top 630 or portions of the first frustum bottom 120 near the second frustum top 630 touching her body as she inserts the applicator 10 into her body. The second frustum 610 and first frustum 110 oriented as shown in FIG. 22 can act as a cushion against the force applied to the insertion member 20.

The second frustum 610 can be a conical frustum. The second frustum 610 can be selected from the group consisting of a spherical frustum, part of an oblate spheroid, part of a prolate spheroid, part of an elliptic cone, part of an ellipsoid, part of a pseudosphere, part of a funnel, part of a hyperboloid, part of a lemon, part of a paraboloid, and a pyramidal frustum.

The second frustum slope 950 for a conical frustum is a straight line. The second frustum slope 950 can be a curved shape corresponding to a defined geometric shape recognized by a person skilled in the art of geometry or can have a curved shape that does not correspond to a shape recognized by a person skilled in the art of geometry.

The second frustum slope 950 can have an irregular shape that is a combination of curved and straight segments, a combination of curved segments having different curvature, or a combination straight segments that are not parallel to adjacent segments.

The second frustum slope 950 is a curved line for a second frustum 610 wherein the second frustum 610 is part of a sphere, part of an oblate spheroid, part of a prolate spheroid, part of an elliptic cone, part of an ellipsoid, part of a pseudosphere, part of a funnel, part of a hyperboloid, part of a lemon, and part of a paraboloid.

The second frustum 610 can have at least one structural discontinuity. The structural discontinuities in the second frustum 610 can be selected from the group consisting of slits, slots, perforations, zones of varied modulus, and apertures. The structural discontinuities for the second frustum 610 can be the same structures as those discussed previously with respect to the first frustum 110.

FIGS. 24, 25, and 26 illustrate an indicator member 100 having a first position, second position, and a third position, in each position, respectively. The object 15 contained within the insertion member 20 can be expelled into the cavity 5 when the indicator member 100 is in any one of the positions. In the first position, an embodiment of which is illustrated in FIG. 24, the tactile signal that the applicator 10 is in a proper position can be the second frustum bottom 620 contacting the boundary of the cavity 5.

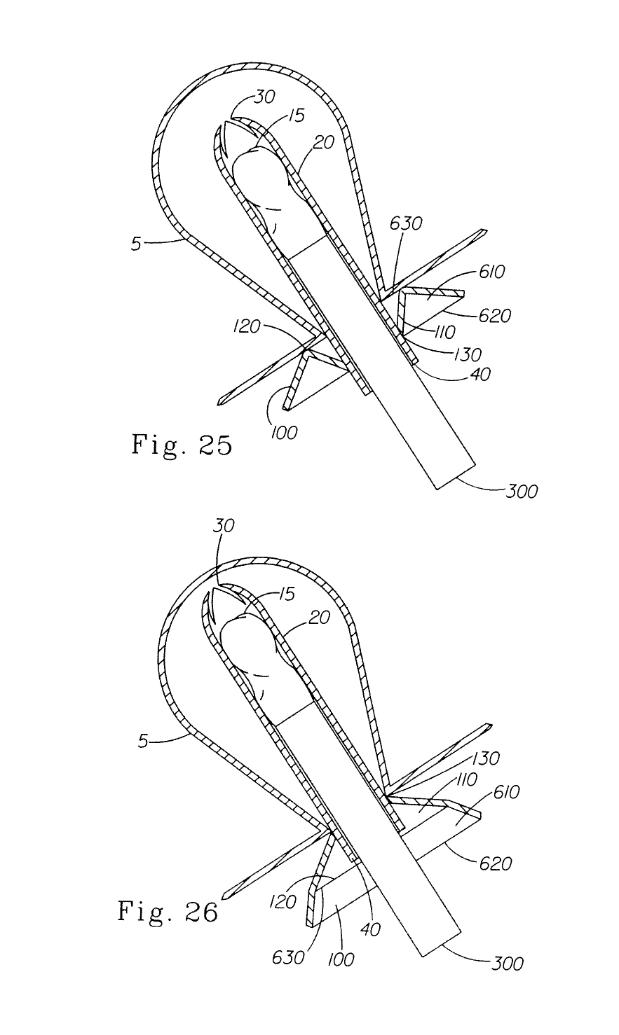

The tactile signal that the applicator 10 is in the proper position can be the indicator member 100 transitioning from a first position, as illustrated in FIG. 24, to a second position, illustrated in FIG. 25. The tactile signal that the applicator 10 is in the proper position can be portions of the first frustum bottom 120 and/or second frustum top 630 contacting the boundaries of the cavity. As discussed above, an indicator member 100 in this configuration can act as a mechanical spring that cushions the boundaries of the cavity from force applied to the insertion member and/or plunger.

The tactile signal that the applicator 10 is in the proper position can be the indicator member 100 transitioning from a second position, as illustrated in FIG. 25, to a third position, illustrated in FIG. 26. The tactile signal that the applicator 10 is in the proper position can be portions of the first frustum top 130 contacting the boundaries of the cavity. As discussed above, an indicator member 100 in this configuration can act as a mechanical spring that cushions the boundaries of the cavity from force applied to the insertion member and/or plunger.

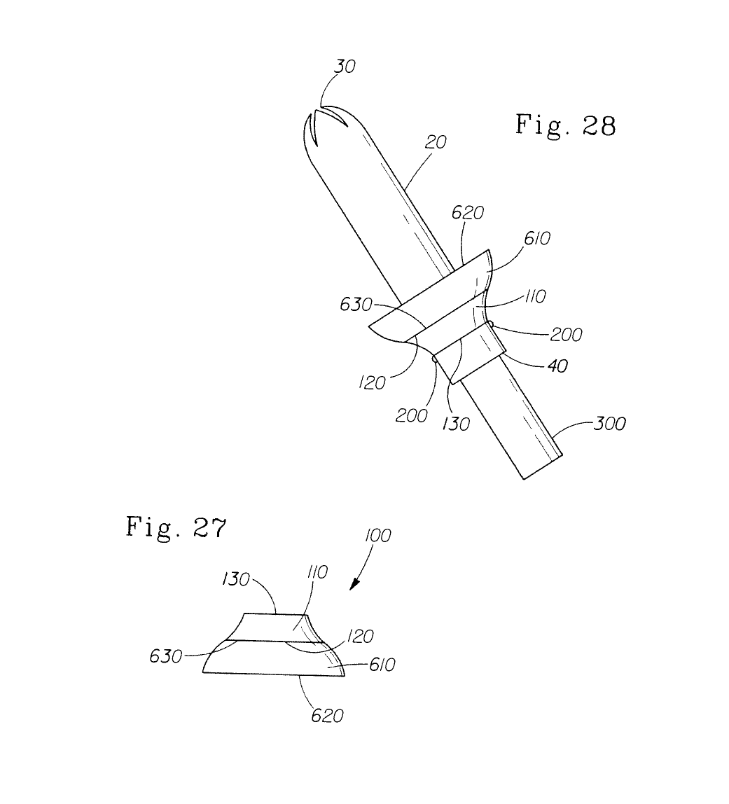

In one embodiment, the indicator member 100 can have the shape of a nipple for a baby bottle from which the dispensing tip of the nipple is cut off, as shown in FIG. 27 in profile. An embodiment of an applicator 10 comprising an indicator member 100 having the shape of a nipple for a baby bottle is shown in FIG. 28.

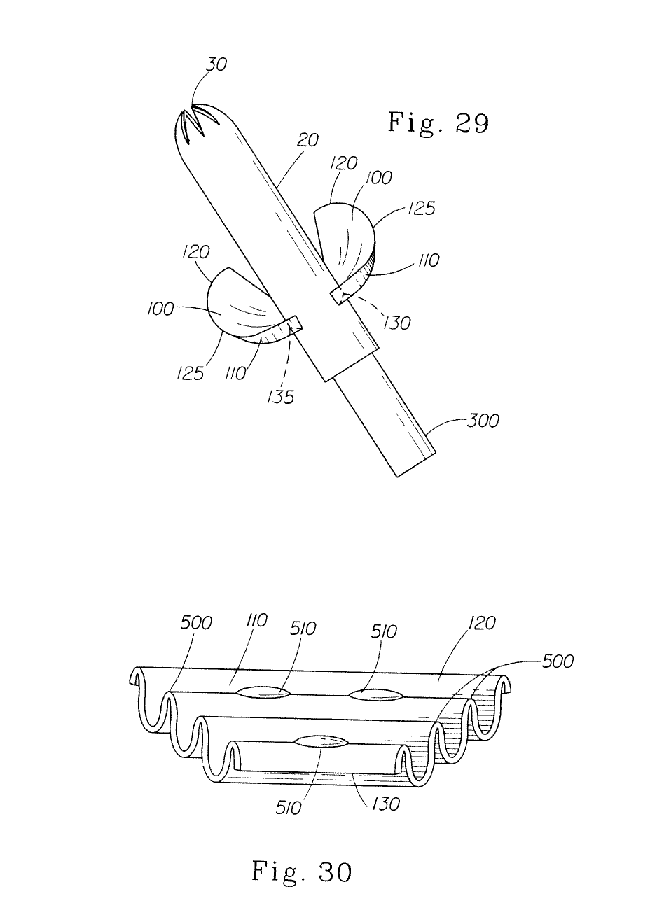

An embodiment in which the indicator member 100 partially circumscribes the periphery of the insertion member 20 is illustrated in FIG. 29. As shown in FIG. 29, the indicator member 100 can comprise part of a hollow open ended first frustum 110.

In embodiments in which the indicator member 100 partially circumscribes the periphery of the insertion member 20, the descriptors for the first frustum 110, including the first frustum top 130, first frustum bottom 120, first frustum top perimeter 135, first frustum bottom perimeter 125, as well as other geometric aspects of the first frustum 110, correspond to the parts and dimensions of a complete frustum.

For instance, if the indicator member 100 is a quadrant of a conical frustum, i.e. one radial fourth of a conical frustum or a portion corresponding to π/2 radians out of the 2π radians encompassing the entire conical frustum, the first frustum top perimeter 135 corresponds to the top perimeter of the entire conical frustum from which the part of the frustum was taken. Thus, for an indicator member 100 comprised of a quadrant of a conical frustum, the entire conical frustum having a perimeter of 100 mm about 2π radians of the conical frustum, the length of the portion of the perimeter of the first frustum 110 employed would be 25 mm (100 mm divided by 4). Similarly, references to the top and bottom refer to the top and bottom of a whole frustum from which the part of the frustum is taken.

A cross section of an embodiment of a first frustum 110 comprised of a stiff material rendered extensible by providing a plurality of corrugations 500 and openable voids 510 is shown in FIG. 30.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An applicator comprising:
a hollow insertion member, said insertion member having an insertion end and an opposing gripper end, a length, and a periphery, the length of said insertion member defined by the distance between said insertion end and said gripper end, wherein the shape defined by the periphery about said insertion member is generally orthogonal to said length of said insertion member; and
an indicator member circumscribing said periphery of said insertion member, said indicator member comprising:
a hollow open ended first frustum, said hollow open ended first frustum having a first frustum bottom, an opposing first frustum top, said first frustum bottom having a first frustum bottom perimeter and said first frustum top having a first frustum top perimeter, said first frustum bottom perimeter greater than said first frustum top perimeter, said first frustum top engaged with said insertion member.

2. An applicator according to claim 1 further comprising a seating member engaged with said insertion member, said first frustum top perimeter engaged with said insertion member through said seating member.

3. An applicator according to claim 1, wherein said first frustum bottom is oriented towards said insertion end of said insertion member.

4. An applicator according to claim 1, wherein said first frustum bottom is oriented away from said insertion end of said insertion member.

5. An applicator according to claim 1, wherein said indicator member has a first position and a second position, wherein in said first position said first frustum bottom is oriented towards said insertion end of said insertion member and in said second position said first frustum bottom is oriented away from said insertion end of said insertion member.

6. An applicator according to claim 1, wherein said first frustum is a conical frustum.

7. An applicator according to claim 1, wherein said first frustum has a curved slope.

8. An applicator according to claim 1 wherein said first frustum comprises at least one structural discontinuity.

9. An applicator according to claim 1 wherein said indicator member is reusable.

10. An applicator according to claim 1 wherein said first frustum is comprised of a flexible material.

11. An applicator according to claim 1 wherein said first frustum is comprised of an extensible material.

12. An applicator according to claim 1, wherein said applicator is an applicator for an article selected from the group consisting of a tampon, a vaginal pessary, a rectal suppository, a vaginally administered medicine, and a vaginally administered contraceptive.

13. An applicator according to claim 1, wherein said indicator member comprises a hollow open ended second frustum having a second frustum bottom, a second frustum top, and a second frustum slope, said second frustum bottom having a second frustum bottom perimeter and said second frustum top having a second frustum top perimeter, said second frustum bottom perimeter greater than said second frustum top perimeter and said second frustum top perimeter equal to said first frustum bottom perimeter, said first frustum bottom perimeter engaged with said second frustum top perimeter, wherein said first frustum has a first frustum slope and said first frustum slope differs from said second frustum slope.

14. An applicator according to claim 13, wherein said second frustum bottom is oriented towards said insertion end of said insertion member.

15. An applicator according to claim 13, wherein said second frustum bottom is oriented away from said insertion end of said insertion member.

16. An applicator according to claim 13, wherein said first frustum bottom is oriented towards said insertion end and said second frustum bottom is oriented away from said insertion end of said insertion member.

17. An applicator according to claim 13, wherein said first frustum bottom is oriented away from said insertion end and said second frustum bottom is oriented away from said insertion end of said insertion member.

18. An applicator according to claim 13 wherein said second frustum has at least one structural discontinuity.

19. An applicator comprising:
a hollow insertion member, said insertion member having an insertion end and an opposing gripper end, a length, and a periphery, the length of said insertion member defined by the distance between said insertion end and said gripper end, wherein the shape defined by the periphery about said insertion member is generally orthogonal to said length of said insertion member; and
an indicator member circumscribing said periphery of said insertion member, said indicator member comprising:
a hollow open ended first frustum, said hollow open ended first frustum having a first frustum bottom, an opposing first frustum top, and a first frustum slope, said first frustum bottom having a first frustum bottom perimeter and said first frustum top having a first frustum top perimeter, said first frustum bottom perimeter greater than said first frustum top perimeter, said first frustum top engaged with said insertion member; and
a hollow insertion member, said insertion member having an insertion end and an opposing gripper end, a length, and a periphery, the length of said insertion member defined by the distance between said insertion end and said gripper end and the shape defined by the periphery about said insertion member being generally orthogonal to said length of said insertion member; and
a hollow open ended second frustum having a second frustum bottom, a second frustum top, and a second frustum slope, said second frustum bottom having a second frustum bottom perimeter and said second frustum top having a second frustum top perimeter, said second frustum bottom perimeter greater than said second frustum top perimeter and said second frustum top perimeter equal to said first frustum bottom perimeter, said first frustum bottom perimeter engaged with said second frustum top perimeter;
wherein said first frustum slope differs from said second frustum slope, wherein said indicator member is comprised of a flexible material and said applicator is sized and dimensioned for insertion into a woman's vagina, wherein said indicator member has a first position and a second position, wherein in said first position said first frustum bottom is oriented towards said insertion end of said insertion member and in said second position said first frustum bottom is oriented away from said insertion end of said insertion member.

20. An applicator comprising:
a hollow insertion member, said insertion member having an insertion end and an opposing gripper end, a length, and a periphery, the length of said insertion member defined by the distance between said insertion end and said gripper end, wherein the shape defined by the periphery about said insertion member is generally orthogonal to said length of said insertion member; and
an indicator member circumscribing said periphery of said insertion member, said indicator member comprising:
a hollow open ended first frustum, said hollow open ended first frustum having a first frustum bottom, an opposing first frustum top, said first frustum bottom having a first frustum bottom perimeter and said first frustum top having a first frustum top perimeter, said first frustum bottom perimeter greater than said first frustum top perimeter, said first frustum top engaged with said insertion member;
wherein said indicator member is comprised of a flexible material and said applicator is sized and dimensioned for insertion into a woman's vagina, wherein said indicator member has a first position and a second position, wherein in said first position said first frustum bottom is oriented towards said insertion end of said insertion member and in said second position said first frustum bottom is oriented away from said insertion end of said insertion member.
